(19) United States
(12) Patent Application Publication
(10) Pub. No.: US 2023/0329559 A1
(43) Pub. Date: Oct. 19, 2023

(54) TRANSCRANIAL MR-GUIDED HISTOTRIPSY SYSTEMS AND METHODS

(71) Applicant: THE REGENTS OF THE UNIVERSITY OF MICHIGAN, Ann Arbor, MI (US)

(72) Inventors: Zhen XU, Ann Arbor, MI (US); Douglas NOLL, Ann Arbor, MI (US); Timothy L. HALL, Ann Arbor, MI (US)

(21) Appl. No.: 18/044,867

(22) PCT Filed: Sep. 13, 2021
(86) PCT No.: PCT/US2021/050088

§ 371 Date: Mar. 10, 2023

Related U.S. Application Data
(60) Provisional application No. 63/078,166, filed on Sep. 14, 2020.
(60) Provisional application No. 63/077,440, filed on Sep. 11, 2020.

(51) Int. Cl. A61N 7/00; A61B 90/00; A61B 5/00; A61B 5/055

(52) U.S. Cl. CPC A61B 5/0036; A61B 5/055; A61B 90/37; A61B 90/39; A61N 7/00; A61B 34/30

(57) ABSTRACT

A transcranial magnetic resonance (MR)-guided histotripsy (tcMRgHt) system is provided. Using electronic steering only, the tcMRgHt system is configured to create lesions of 25.5 mm in the transverse plane and 50 mm in the axial plane through the skull of a patient. This disclosure provides the design, fabrication, acoustic characterization, and MR compatibility assessment of tcMRgHt systems for histotripsy.

### CROSS REFERENCE TO RELATED APPLICATIONS

This application claims the benefit of U.S. Provisional Application No. 63/077,440, filed Sep. 11, 2020, entitled “Transcranial MR-Guided Histotripsy Systems and Methods”, and of U.S. Provisional Application No. 63/078,166, filed Sep. 14, 2020, entitled “Transcranial MR-Guided Histotripsy Systems and Methods”, each of which is herein incorporated by reference in its entirety.

### INCORPORATION BY REFERENCE

U.S. Application No. 16/698,587, filed Nov. 27, 2019, entitled “Histotripsy Systems and Methods”, and U.S. Application No. 15/737,761, filed Dec. 19, 2017, entitled “Histotripsy Therapy Systems and Methods for the Treatment of Brain Tissue” are incorporated by reference herein.

All publications and patent applications mentioned in this specification are herein incorporated by reference to the same extent as if each individual publication or patent application was specifically and individually indicated to be incorporated by reference.

### STATEMENT AS TO FEDERALLY SPONSORED RESEARCH

This invention was made with Government support under R01 EB028309 awarded by the National Institutes of Health. The Government has certain rights in the invention.

### FIELD

The present disclosure details novel histotripsy systems configured to produce acoustic cavitation, methods, devices and procedures for the minimally and non-invasive treatment of healthy, diseased and/or injured tissue. The histotripsy systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Many medical conditions require invasive surgical interventions. Invasive procedures often involve incisions, trauma to muscles, nerves and tissues, bleeding, scarring, trauma to organs, pain, need for narcotics during and following procedures, hospital stays, and risks of infection. Non-invasive and minimally invasive procedures are often favored, if available, to avoid or reduce such issues. Unfortunately, non-invasive and minimally invasive procedures may lack the precision, efficacy or safety required for treatment of many types of diseases and conditions. Enhanced non-invasive and minimally invasive procedures are needed, preferably not requiring ionizing or thermal energy for therapeutic effect.

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, and high-intensity focused ultrasound (HIFU), Histotripsy relies on the mechanical action of cavitation for tissue destruction.

An important recent trend in medical interventions is a comprehensive drive towards less invasive yet effective procedures. Many disease states can now be addressed using minimally-invasive or non-invasive approaches, and many of these are performed under increasingly sophisticated imaging guidance. The progression from planar radiation therapy to stereotactic body radiation therapy (SBRT) is one such example, but radiation toxicity still limits treatment locations and volume. Thermal-based ablations are generally delivered percutaneously with imaging guidance, and include radiofrequency ablation, microwave ablation, and cryoablation. These technologies either heat or freeze targeted tissue which results in necrosis. All thermal modalities are impacted by the heat sink effect of blood flow, a critical reliance on physician expertise, tumor size, tumor location, and a lack of predictability of the ablation margins. High intensity focused ultrasound (HIFU) is a non-invasive ablation technique that uses externally applied ultrasound energy to cause thermal necrosis. HIFU has been used clinically to treat uterine fibroids, neurological diseases, and tumors in the prostate, breast, liver, and pancreas, but its clinical use is still infrequent due to anatomic challenges and long procedure times.

Histotripsy is a non-invasive focused ultrasound technology that uses ultrasound applied from outside the body and focused on a target tissue. The underlying mechanism of histotripsy is mechanical at the cellular level, which is entirely different from HIFU thermal therapy. The term histotripsy was coined at the University of Michigan in 2003. In Greek, “Histo” means “soft tissue,” and “tripsy” refers to breakdown. HIFU uses continuous or long exposure of ultrasound with intermediate applied pressure and high duty cycles (ultrasound on-time/total treatment time 10%) to heat target tissue. In contrast, histotripsy uses a low duty cycle (1%) to minimize heating, short ultrasound pulses (microseconds to milliseconds in length), and very high applied pressure to generate acoustic cavitation using endogenous gas in tissues. Acoustic cavitation is the initiation and dynamic changes of microbubbles activated by ultrasound. Histotripsy uses cavitation to mechanically break down and liquefy the target tissue into an acellular debris. Ultrasound imaging can be used to guide and monitor the histotripsy procedure in real time. In contrast to many existing minimally-invasive techniques, histotripsy can result in the non-invasive removal of tissue. When histotripsy is applied to a tissue-fluid interface (e.g., blood clots or cardiac tissue), tissue is eroded from the surface inwards, and eventually results in well-defined perforations. When targeting histotripsy to inside a bulk volume tissue (e.g., a tumor), histotripsy eventually liquifies the target tissue to an acellular homogenate, and the debris is absorbed over 1-3 months by the body, resulting in effective tissue removal.

The ability to effectively remove tissue allows histotripsy to be used in applications that are not possible with thermal techniques. The non-thermal nature also enables histotripsy to overcome many of the limitations associated with thermal devices (e.g., heat sink effect, lack of precise margins and predictability). Histotripsy has been investigated for many pre-clinical applications, including treatment for tumors in the liver, kidney, and prostate, neurological diseases, thrombosis, neonatal and fetal congenital heart disease, kidney stones, and biofilms. Phase I human trials have been undertaken for histotripsy treatment of benign prostatic hyperplasia and liver cancer, and early results suggest safety and feasibility in humans. This review provides a comprehensive overview of histotripsy, including the mechanism, bioeffects, parameters, instruments, preclinical and clinical studies, and advantages and limitations compared to related devices.

For a non-invasive therapy, imaging guidance is crucial to ensure high treatment accuracy. Ultrasound imaging is typically used to guide the targeting and monitoring for histotripsy treatment, as cavitation can be clearly visualized on clinical B-mode as temporally changing, hyperechoic (bright) zone. An ultrasound imaging probe is co-aligned with the histotripsy focal zone to imaging the focal zone. The focal position is marked on the 2D B-mode ultrasound image. Ultrasound imaging is scanned over a 3D volume to identify the treatment target (e.g., a tumor), and then the focal location marked on B-mode ultrasound is aligned with the target. During the treatment, the temporally changing hyperechoic cavitation zone grows larger with more flickering motion as the target volume tissue is gradually fractionated into a liquefied homogenate. The completed liquefied tissue appeared as a hypoechoic (dark) zone on ultrasound imaging, because the number and size of the sound scatterers reduce significantly when the tissue is liquefied to acellular debris. The hypoechoic zone can be used to determine the treatment completion. Histotripsy ablation zone can also be monitored by ultrasound elastography, as it becomes gradually softer and eventually liquefied.

There are two main limitations for ultrasound imaging guidance. First, certain tumors can be seen clearly on MRI or CT, but not on ultrasound. In these cases, it is possible to co-register and/or fuse ultrasound images with pre-treatment MRI or CT scans to increase the targeting accuracy. Second, 2D ultrasound cannot provide 3D volume imaging during treatment. 2D ultrasound imaging is used for histotripsy guidance, because the footprint of a 3D ultrasound imaging probe is much larger, occupying the acoustic window space needed for the histotripsy transducer.

MRI is a primary clinical tool for tumor imaging. MRI can provide high tumor-tissue contrast and also visualize soft tissue well in general. MRI also provides 3D volumetric imaging. MRI can be used to guide histotripsy treatment to overcome the limitations of ultrasound imaging as described above. Particularly, ultrasound cannot be used for brain imaging due to the skull blockage. MRI is a routine clinical tool for brain imaging to diagnose brain tumors, stroke, and neurological diseases as well as evaluate brain injuries. Therefore, for histotripsy brain treatment, MR guidance is necessary.

MR-guided focused ultrasound (MRgFUS) - MRI has been developed and used to guide high intensity focused ultrasound (HIFU) thermal therapy in various preclinical and clinical work, as MR thermometry can be used to image the temperature change generated by HIFU. As a matter of fact, for HIFU, MRI guidance is preferred over ultrasound imaging, even with the high cost, because temperature change cannot be visualized by ultrasound imaging. MR-guided focused ultrasound (MRgFUS) has been used to treat benign and malignant tumors, including uterine fibroids, prostate cancer, liver cancer, etc. MR thermometry is used to measure the temperature increase during HIFU to guide the targeting and monitoring of the HIFU treatment. For targeting, HIFU is used to increase the temperature at the focus to 1-4deg C, which is below the biological damage level, but this low temperature increase can be visualized by MR thermometry to identify the HIFU focal location. After targeting confirmation, HIFU treatment is delivered, and MR thermometry is used to monitor the thermal dose delivered to the treatment region in real-time.

Transcranial MR-guided focused ultrasound (tcMRgFUS) - Guided by real-time MRI brain scan, ultrasound is delivered through the skull and focused to the target brain tissue. TcMRgFUS can be used to deliver high intensity continuous ultrasound wave or long pulses to create thermal ablation in the brain, open blood brain barrier (BBB) for drug delivery, and neural stimulation. In July 2016, the U.S. Food and Drug Administration (FDA) approved a tcMRgFUS device (ExAblate Neuro, InSightec) to treat essential tremor. Clinical trials are ongoing to investigate tcMRgFUS for treatment Parkinson’s disease, Alzheimer’s disease, and brain tumor. However, tcMRgFUS thermal ablation has fundamental limitations on treatment location and volume, due to overheating of the skull, which is both highly absorptive and reflective of ultrasound. 1) The treatment location profile is limited mostly to the central region of the brain, while approximately 90% of the brain cortex region (<2 cm from the skull surface), where tumors often reside, cannot be treated. 2) tcMRgFUS cannot treat a volume target (>1 cm diameter) in a reasonable time. These two limitations on the treatment location and volume are major roadblocks, preventing the widespread use of tcMRgFUS to treat brain tumors.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIGS. 2A-2B illustrate a MR-compatible histotripsy transducer array.

FIGS. 3A-3C show the cross-section of transducer module design and photo of an element fabricated in house.

FIGS. 4A-4B show an exploded view (A) and photo (B) of a Histotripsy Array and Accessories.

FIGS. 5A-5C show an experimental setup. Diagram (A) and photo (B) of the experimental setup for in vivo pig treatment. MR images on transverse plane (C) demonstrate the positions of transducer elements, human skull and pig brain.

FIG. 6 shows 1-D beam profiles around the geometric focus.

FIG. 7 shows the -6 dB range in three cases remained almost identical, but the effective therapeutic range (where p->26 MPa can be achieved) through the skull was significantly smaller than that in free field, because the attenuation and aberration induced by the skullcap became prominent as the steering locations move further from the geometric focus.

FIGS. 8A-8B show histotripsy ablation generated by electronic focal steering in the RBC phantom. A: Sparse circular pattern centered at geometric focus. B: A 10-mm continuous square lesion representing a volume ablation zone in transverse plane.

FIGS. 9A-9D show the images acquired from four cases described herein and their corresponding SNR.

FIGS. 10A-10C show B0 and B1 field maps for tcMRgHt experiments. (A): The binary mask applied to reconstructed field maps. (B): B0 field map for off-resonance in Hz. (C): B1 field map for actual tip angle.

FIGS. 11A-11G show T2-weighted MR images showed hyper-intense histotripsy ablation zones compared to the surrounding untreated tissue, as the cavitation generated by histotripsy liquefies the tissue.

### SUMMARY OF THE DISCLOSURE

Histotripsy produces tissue fractionation through dense energetic bubble clouds generated by short, high-pressure, ultrasound pulses. When using pulses shorter than 2 cycles, the generation of these energetic bubble clouds only depends on where the peak negative pressure (P-) exceeds an intrinsic threshold for inducing cavitation in a medium (typically 26 -30 MPa in soft tissue with high water content).

In some embodiments, a method of treating a patient with MR-guided histotripsy therapy is provided, comprising the steps of identifying an ultrasound focal location of a histotripsy therapy transducer on a MR image, positioning the ultrasound focal location on a target tissue, transmitting histotripsy pulses from the histotripsy therapy transducer into the target tissue to generate cavitation in the target tissue, and acquiring MR images of the target tissue to monitor cavitation in the target tissue.

In some embodiments, identifying the ultrasound focal location comprises emitting ultrasound energy below a cavitation threshold from the histotripsy therapy transducer, and detecting the ultrasound energy with a MR-ARFI system.

In one embodiment, detecting the ultrasound energy with the MR-ARFI system comprises detecting displacement at the ultrasound focal location.

In another embodiment, identifying the ultrasound focal location comprises emitting ultrasound energy to create a 1-4 deg C temperature increase at the ultrasound focal location, and detecting the temperature increase with a MR thermometry system.

In some examples, the histotripsy pulses are transmitted through a skull of a patient.

In some embodiments, the target tissue is in a brain of a patient.

In one embodiment, acquiring images further comprises acquiring images of bubble expansion and collapse events and not the cavitation itself.

In some embodiments, acquiring MR images of the target tissue further comprises acquiring MR images with an intravoxel incoherent motion (IVIM) imaging pulse sequence.

In one embodiment, the IVIM sequence comprises a spin-echo (SE) sequence.

Other embodiments comprise acquiring MR thermometry images of the target tissue to monitor heating of the target tissue.

In some embodiments, acquiring MR thermometry images is interleaved with acquiring MR images.

In one embodiment, the method further comprises acquiring post-treatment MR images to evaluate histotripsy ablation.

In another embodiment, the method further comprises quantitatively assessing a level of tissue disruption generated by histotripsy with the post-treatment MR images.

In one embodiment, the method further comprises applying diffusion weighted MRI.

In one embodiment, the method further comprises applying MR elastography.

In some examples, the acquiring MR images step is synchronized with the transmitting histotripsy pulses step.

An ultrasound system is provided, comprising a histotripsy therapy transducer configured to transmit histotripsy pulses to an ultrasound focal location in a target tissue volume, and a MRI system configured to generate MR images of the target tissue volume, the MRI system being further configured to identify the ultrasound focal location of on a MR image of the target tissue volume, the MRI system being further configured to acquire MR images of the target tissue to monitor cavitation resulting from the histotripsy pulses in the target tissue.

### DETAILED DESCRIPTION

Provided herein are systems and methods that provide efficacious non-invasive and minimally invasive therapeutic, diagnostic and research procedures. In particular, provided herein are optimized systems and methods that provide targeted, efficacious histotripsy in a variety of different regions and under a variety of different conditions without causing undesired tissue damage to intervening/non-target tissues or structures.

Balancing desired tissue destruction in target regions with the avoidance of damage to non-target regions presents a technical challenge. This is particularly the case where time efficient procedures are desired. Conditions that provide fast, efficacious tissue destruction tend to cause undue heating in non-target tissues. Under heating can be avoided by reducing energy or slower delivery of energy, both of which run contrary to the goals of providing a fast and efficacious destruction of target tissue. Provided herein are a number of technologies that individually and collectively allow for fast, efficacious target treatment without undesired damage to non-target regions.

The system, methods and devices of the disclosure may be used for the minimally or non-invasive acoustic cavitation and treatment of healthy, diseased and/or injured tissue, including in extracorporeal, percutaneous, endoscopic, laparoscopic, and/or as integrated into a robotically-enabled medical system and procedures. As will be described below, the histotripsy system may include various electrical, mechanical and software sub-systems, including a Cart, Therapy, Integrated Imaging, Robotics, Coupling and Software. The system also may comprise various Other Components, Ancillaries and Accessories, including but not limited to patient surfaces, tables or beds, computers, cables and connectors, networking devices, power supplies, displays, drawers/storage, doors, wheels, illumination and lighting and various simulation and training tools, etc. All systems, methods and means creating/controlling/delivering histotripsy are considered to be a part of this disclosure, including new related inventions disclosed herein.

This disclosure describes devices and methods of MR-guided histotripsy (MRgHt), including the MR-conditional histotripsy system, the specialized MRI pulse sequence for histotripsy treatment guidance and monitoring, and the MRgHt workflow.

MR-guided histotripsy (MRgHt) - MRI can be used to guide the histotripsy treatment. Similar to MRgFUS, MRgHt uses MRI to guide the delivery of focused ultrasound to the target tissue and requires a MR-compatible ultrasound transducer. But MRgHt is different from MRgFUS mainly in two ways. 1) In contrast to the continuous ultrasound wave or long ultrasound pulses used in MRgFUS, Histotripsy uses micro-second length ultrasound pulses at very high pressure (p->20 MPa) to generate focal cavitation, thus the ultrasound transducer and associated driving electronics used in MRgHt are substantially different from those used in MRgFUS. 2) As histotripsy generates cavitation rather than heating the tissue, MR thermometry cannot be used to monitor the histotripsy treatment, specialized MRI pulse sequence has to be developed to enable real-time cavitation monitoring. Such MRI imaging sequence is not currently available.

Transcranial MR-guided histotripsy (TcMRgHt) - For TcMRgHt, microsecond pulses with a very low duty cycle (ultrasound on-time/off-time << 0.1%) is used to minimize the skull heating. Preliminary studies show that transcranial histotripsy can be used to treat a wide range of locations and volumes through an excised human skull without overheating the skull thus potentially overcoming the treatment location and volume limitation of tcMRgFUS. The initial in vivo safety of histotripsy brain treatment was demonstrated in the in vivo normal porcine brain, where no excessive bleeding or hemorrhage or other brain injury was generated outside the targeted ablation zone. TcMRgHt requires a specialized MRI compatible transcranial histotripsy system that can produce sufficiently high pressure (p->26 MPa), 1-cycle pulses through a human skull. Considering the high attenuation and aberration caused by ultrasound propagation through the skull, such a transcranial histotripsy system is highly technically challenging and innovative.

MR-conditional histotripsy system - An MR-conditional histotripsy system contains an ultrasound histotripsy transducer, the associated electronic driver, and cables connecting the two with the following main innovative features. MR-conditional for purposes of this disclosure means MR compatible under particular operating conditions.

The histotripsy system is capable of generating microsecond-length pulses and high pressure (p->20 MPa). This is enabled by a focused ultrasound transducer with a high bandwidth and an electronic driver that can produce >1 kV bursts to drive the ultrasound transducer.

All metallic components in the ultrasound histotripsy transducer and associated cables and interconnects include only non-ferrous materials. The mass of metal also needs to be minimized to maintain good MR image quality. Specifically, segmented ground plane rather than continuous ground plane is used to allow RF magnetic field penetration through spaces between the ground plane.

As the histotripsy electronic driver contains ferrous material, the electronic driver is placed outside the MRI room. For example, the electronic driver can be placed in the adjacent equipment room or in a RF-shielded container away from the main magnetic field by placing the cables through wave guide penetrations or through RF-filtered connections.

The ultrasound transducer housing can have MR fiducial markers that can be used to localize the geometric focus of the transducer on MR images.

The driver cables break the RF shielding of the MR system by passing through the barrier. This reduces the SNR of the MR system degrading image quality. In some embodiments, filtered connections or floating cable traps can be used to prevent noise leakage and maintain the MR imaging quality. Alternatively, the coaxial cable shields can be connected together at the point where they pass through the barrier and attached to the room shield (ground).

To enable MR-ARFI or MR thermometry, a hybrid electronic driver is required which is capable of producing continuous excitation at lower amplitude as well as short very intense bursts at high amplitude for histotripsy.

MRI pulse sequence for histotripsy guidance and monitoring - HIFU produces heating to cause tissue necrosis, and thus MR thermometry is typically used in HIFU applications to guide and monitor the HIFU treatment. In contrast, histotripsy generates cavitation that mechanically breaks down the target tissue and eventually liquefies the tissue to acellular debris. Therefore, new specialized MRI pulse is needed to monitor cavitation and the tissue disruption generated by histotripsy, for pre-treatment targeting, monitoring during treatment, and post-treatment tissue evaluation.

Pre-treatment targeting - For pre-treatment targeting, the ultrasound focal location must be identified on MRI without generating cavitation which damaged tissues. This can be achieved by MR-ARFI (acoustic radiation force impulse), MR thermometry with low temperature heating, or MR fiducial markers, as described below.

For MR-ARFI, the transducer emits ultrasound below the cavitation threshold to generate acoustic radiation force and subsequent displacement at the ultrasound focus without generating cavitation. MRI can then be used to detect the displacement to identify the ultrasound focus location on MR images via displacement encoding gradients. Phase images are compared to a control with no ultrasound or displacement applied in an opposite direction. One manifestation uses trains of short, low amplitude ultrasound pulses in lieu of continuous wave ultrasounds pulses in some implementations of MR-ARFI.

MR thermometry - The transducer can emit ultrasound to create 1-4deg C temperature increase, which is sufficiently low and would not create any damage. MR thermometry is then used to detect the temperature increase and identify the location of maximum temperature increase as the ultrasound focus on MR images. One manifestation uses trains of short, low amplitude ultrasound pulses in lieu of continuous wave ultrasounds pulses in some implementations of MR thermometry.

MR fiducial markers - MR fiducial markers on the ultrasound transducer housing can allow the identification of the geometry location of the ultrasound transducer on MR images. However, if the focal location shifts from the geometric focus due to acoustic aberration, this method won’t be able to detect the focal shift.

Once the ultrasound focal location is identified on the MR images, the ultrasound transducer focus is moved mechanically or electronically to be aligned on the target tissue. Histotripsy treatment can be initiated when the ultrasound focal location is positioned on the target tissue. The treatment can include transmitting histotripsy ultrasound pulses from the histotripsy system (or ultrasound transducer) into the target tissue to generate cavitation in the target tissue.

Procedural Monitoring - For procedural imaging, histotripsy-induced cavitation can be visible on MRI with special pulse-sequences synchronized to the ultrasound therapy or histotripsy pulses, resulting in real-time MRI monitoring. MRI bubble imaging methods based on either T2^* contrast or cavity void detection generally cannot be used for histotripsy cavitation because they require bubbles to occupy a large fraction of the voxel volume and to be present for an extended period of time, whereas histotripsy bubbles occupy only a small fraction of the voxel volume and last for microseconds. Therefore, for procedural MRI imaging of histotripsy cavitation, according to this disclosure, the MRI can be configured to image the localized, random flow due to bubble expansion and collapse events and not the bubble itself with an intravoxel incoherent motion (IVIM) imaging pulse sequence. The IVIM pulse sequence uses histotripsy-synchronized, displacement encoding gradients to induce random phases associated with random flow, leading to MRI signal reduction in response to histotripsy pulses. In one embodiment, the IVIM pulse sequence can be a spin-echo (SE) sequence rather than a gradient-echo (GRE), with an optimized IVIM gradient b-value as well as trigger sent between the MR-scanner and the histotripsy transducer to synchronize the two. In some embodiments, cavitation detection can be interleaved with MR thermometry to simultaneously monitor heating of tissues during treatment. Multiple ultrasound therapy pulses can be applied during the encoding interval to induce more flow within a voxel and/or extend into additional imaging voxels.

Post-treatment evaluation - MRI can be used to evaluate the post-treatment effect of histotripsy. Histotripsy ablation zones are clearly visualized on T1-weighted, T2-weighted, T2*-weighted, and contrast-enhanced MR images. For example, on T1-weighted images, ablation zones are hypointense due to retained blood products. After contrast, ablation zones do not enhance, allowing differentiation of the ablation zone from residual tumor. In addition, as histotripsy mechanically breaks down the target tissue, the tissue becomes softer gradually and is eventually liquefied. MR elastography and diffusion-weighted MRI can also be used to evaluate the histotripsy tissue effect after treatment and potentially during treatment.

Diffusion-weighted MRI created images of the diffusion of water. When histotripsy breaks cell membranes, nuclear and other subcellular structures, water diffusion is no longer hindered by these structures resulting in greater diffusion, which can be visualized using diffusion-weighted MRI. The increase in diffusion results in decreased image intensity in diffusion-weighted MR images or increases in the calculated diffusion coefficient. The diffusion effects are related to the completeness of tissue ablation and thus can be used to monitor or assess degree of treatment.

MRgHt workflow - There are five steps in the MRgHt workflow.

The patient lays on the MRI bed, and the ultrasound transducer is coupled to the patient, likely with a water bath coupling.

Prior to the treatment, the patient is imaged in the MRI scanner, and MR images will be used to identify the target tissue.

One or more pre-treatment targeting methods described above is used to identify the ultrasound focal location. The ultrasound transducer focus is moved mechanically or electronically to align the ultrasound focus on the target tissue on MR images. A treatment location grid can be created to paint the ultrasound focus over the target volume (i.e. a tumor volume).

Once the targeting is confirmed, histotripsy treatment with preset ultrasound parameters is delivered to the target volume. In some embodiments, aberration correction of the therapy pulses may be applied before treatment application to correct any aberration due to ultrasound propagating through the overlying tissue including bones. Aberration correction is particularly needed for brain applications due to aberrations caused by the skull. In some embodiments, the histotripsy system includes both transmit and receive capabilities, so that the aberrations can be detected and accounted for with the aberration correction algorithm. Additional details on a histotripsy system having transmit-receive capabilities are described in International Patent Application No. PCT/US2021/048008, filed Aug. 27, 2021. The procedural monitoring method(s) described above and herein can be used to monitor cavitation during treatment to ensure that the treatment locations are all within the target tissue volume.

After the treatment is delivered, post-treatment evaluation methods(s) described above can be used to assess whether the desired tissue effect is achieved. If not, additional treatment can be delivered until the desired tissue effect is achieved, and the treatment is complete.

In one embodiment, the histotripsy system is configured as a mobile therapy cart, which further includes a touchscreen display with an integrated control panel with a set of physical controls, a robotic arm, a therapy head positioned on the distal end of the robot, a patient coupling system and software to operate and control the system.

The mobile therapy cart architecture can comprise internal components, housed in a standard rack mount frame, including a histotripsy therapy generator, high voltage power supply, transformer, power distribution, robot controller, computer, router and modem, and an ultrasound imaging engine. In some embodiments, all components of the mobile therapy cart can be MRI compatible. The front system interface panel can comprise input/output locations for connectors, including those specifically for two ultrasound imaging probes (handheld and probe coaxially mounted in the therapy transducer), a histotripsy therapy transducer, AC power and circuit breaker switches, network connections and a foot pedal. The rear panel of the cart can comprise air inlet vents to direct airflow to air exhaust vents located in the side, top and bottom panels. The side panels of the cart include a holster and support mechanism for holding the handheld imaging probe. The base of the cart can be comprised of a cast base interfacing with the rack mounted electronics and providing an interface to the side panels and top cover. The base also includes four recessed casters with a single total locking mechanism. The top cover of the therapy cart can comprise the robot arm base and interface, and a circumferential handle that follows the contour of the cart body. The cart can have inner mounting features that allow technician access to cart components through access panels.

The touchscreen display and control panel may include user input features including physical controls in the form of six dials, a space mouse and touchpad, an indicator light bar, and an emergency stop, together configured to control imaging and therapy parameters, and the robot. The touchscreen support arm is configured to allow standing and seated positions, and adjustment of the touchscreen orientation and viewing angle. The support arm further can comprise a system level power button and USB and ethernet connectors.

The robotic arm can be mounted to the mobile therapy cart on arm base of sufficient height to allow reach and ease of use positioning the arm in various drive modes into the patient/procedure work space from set up, through the procedure, and take down. The robotic arm can also be MRI compatible. The robotic arm can comprise six degrees of freedom with six rotating joints, a reach of 850 mm and a maximum payload of 5 kg. The arm may be controlled through the histotripsy system software as well as a 12 inch touchscreen polyscope with a graphical user interface. The robot can comprise force sensing and a tool flange, with force (x, y, z) with a range of 50 N, precision of 3.5 N and accuracy of 4.0 N, and torque (x, y, z) with a range of 10.0 Nm, precision of 0.2 Nm and accuracy of 0.3 Nm. The robot has a pose repeatability of +/- 0.03 mm and a typical TCP speed of 1 m/s (39.4 in/s). In one embodiment, the robot control box has multiple I/O ports, including 16 digital in, 16 digital out, 2 analog in, 2 analog out and 4 quadrature digital inputs, and an I/O power supply of 24V/2A. The control box communication comprises 500 Hz control frequency, Modbus TCP, PROFINET, ethernet/IP and USB 2.0 and 3.0.

The therapy head can comprise one of a select group of four or more histotripsy therapy transducers and an ultrasound imaging system/probe, coaxially located in the therapy transducer, with an encoding mechanism configured to rotate the imaging probe independent of the therapy transducer to known positions, and a handle to allow gross and fine positioning of the therapy head, including user inputs for activating the robot (e.g., for free drive positioning). In some examples, the therapy transducers may vary in size (22 × 17 cm to 28 × 17 cm), focal lengths from 12 - 18 cm, number of elements, ranging from 48 to 64 elements, comprised within 12-16 rings, and all with a frequency of 700 kHz. The therapy head subsystem has an interface to the robotic arm includes a quick release mechanism to allow removing and/or changing the therapy head to allow cleaning, replacement and/or selection of an alternative therapy transducer design (e.g., of different number of elements and geometry), and each therapy transducer is electronically keyed for auto-identification in the system software.

The patient coupling system can comprise a six degree of freedom, six joint, mechanical arm, configured with a mounting bracket designed to interface to a surgical/interventional table rail. The arm may have a maximum reach of approximately 850 mm and an average diameter of 50 mm. The distal end of the arm can be configured to interface with an ultrasound medium container, including a frame system and an upper and lower boot. The lower boot is configured to support either a patient contacting film, sealed to patient, or an elastic polymer membrane, both designed to contain ultrasound medium (e.g., degassed water or water mixture), either within the frame and boot and in direct contact with the patient, or within the membrane/boot construct. The lower boot provides, in one example, a top and bottom window of approximately 46 cm × 56 cm and 26 cm × 20 cm, respectively, for placing the therapy transducer with the ultrasound medium container and localized on the patient’s abdomen. The upper boot may be configured to allow the distal end of the robot to interface to the therapy head and/or transducer, and to prevent water leakage/spillage. In preferred embodiments, the upper boot is a sealed system. The frame is also configured, in a sealed system, to allow two-way fluid communication between the ultrasound medium container and an ultrasound medium source (e.g., reservoir or fluidics management system), including, but not limited for filling and draining, as well as air venting for bubble management.

The system software and work-flow can be configured to allow users to control the system through touchscreen display and the physical controls, including but not limited to, ultrasound imaging parameters and therapy parameters. The graphical user interface of the system comprises a work-flow based flow, with the general procedure steps of 1) registering/selecting a patient, 2) planning, comprising imaging the patient (and target location/anatomy) with the freehand imaging probe, and robot assisted imaging with the transducer head for final gross and fine targeting, including contouring the target with a target and margin contour, of which are typically spherical and ellipsoidal in nature, and running a test protocol (e.g., test pulses) including a bubble cloud calibration step, and a series of predetermined locations in the volume to assess cavitation initiation threshold and other patient/target specific parameters (e.g., treatment depth), that together inform a treatment plan accounting for said target’s location and acoustic pathway, and any related blockage (e.g., tissue interfaces, bone, etc.) that may require varied levels of drive amplitude to initiate and maintain histotripsy. Said parameters, as measured as a part of the test protocol, comprising calibration and multi-location test pulses, are configured in the system to provide input/feedback for updating bubble cloud location in space as needed/desired (e.g., appropriately calibrated to target cross-hairs), as well as determining/interpolating required amplitudes across all bubble cloud treatment locations in the treatment volume to ensure threshold is achieved throughout the volume. Further, said parameters, including but not limited to depth and drive voltage, may be also used as part of an embedded treatability matrix or look up table to determine if additional cooling is required (e.g., off-time in addition to time allocated to robot motions between treatment pattern movements) to ensure robust cavitation and intervening/collateral thermal effects are managed (e.g., staying below t43 curve for any known or calculated combination of sequence, pattern and pathway, and target depth/blockage). The work-flow and procedure steps associated with these facets of planning, as implemented in the system software may be automated, wherein the robot and controls system are configured to run through the test protocol and locations autonomously, or semi-autonomously. Following planning, the next phase of the procedure work-flow, 3) the treatment phase, is initiated following the user accepting the treatment plan and initiating the system for treatment. Following this command, the system is configured to deliver treatment autonomously, running the treatment protocol, until the prescribed volumetric treatment is complete. The status of the treatment (and location of the bubble cloud) is displayed in real-time, adjacent to various treatment parameters, including, but not limited to, of which may include total treatment time and remaining treatment time, drive voltage, treatment contours (target/margin) and bubble cloud/point locations, current location in treatment pattern (e.g., slice and column), imaging parameters, and other additional contextual data (e.g., optional DICOM data, force torque data from robot, etc.). Following treatment, the user may use the therapy head probe, and subsequently, the freehand ultrasound probe to review and verify treatment, as controlled/viewed through the system user interface. If additional target locations are desired, the user may plan/treat additional targets, or dock the robot to a home position on the cart if no further treatments are planned.

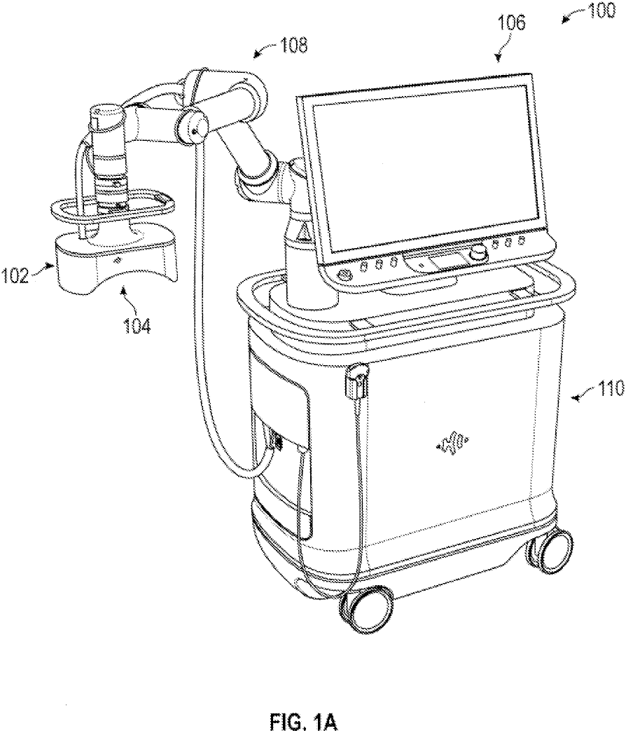

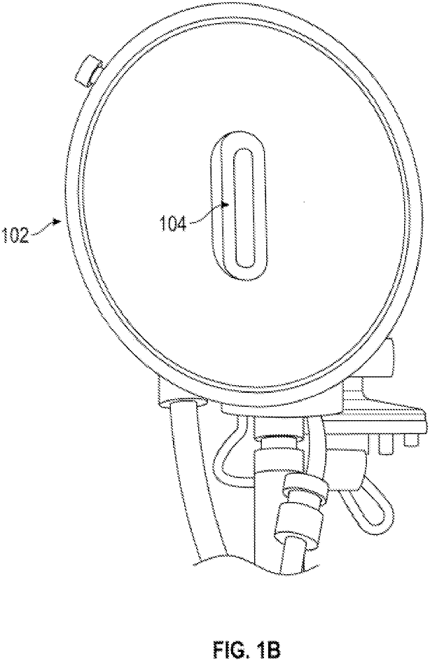

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy).

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with at least a single negative/tensile phase sufficient to cause a cluster of bubble nuclei intrinsic to the medium to undergo inertial cavitation, 2) Shock-Scattering Histotripsy: Delivers typically pulses 3-20 cycles in duration. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26 - 30 MPa for soft tissues with high water content, such as tissues in the human body. In some embodiments, using this intrinsic threshold mechanism, the spatial extent of the lesion may be well-defined and more predictable. With peak negative pressures (P-) not significantly higher than this threshold, sub-wavelength reproducible lesions as small as half of the -6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically < 2 cycles and having a frequency between 100 kHz and 1 MHz) can be applied together with a high-frequency “probe” pulse (typically < 2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P- level for a region of interest (ROI), while the high-frequency pulse, which provides more precision, can pin-point a targeted location within the ROI and raise the peak negative pressure P- above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Trandsducer Array and Fabrication

A key component of histotripsy therapy is a high-power focused ultrasound transducer array configured to deliver sufficiently high ultrasound pressure and power to generate cavitation in the target tissue. Traditional transducer fabrication techniques include heating a large piece of piezoelectric (PZT) or piezoceramic composite (PCC) material and shaping the material to the appropriate curved shape with high mechanical precision. Next, the shaped PZT or PCC material can be cut into individual transducer elements. Electrode connections are then soldered to the individual transducer elements. In some implementations, a thin curved matching layer can then be bonded to the curved PZT or PCC transducer elements. One advantage of the traditional fabrication approach is that the packing density (area occupied by PZT or PCC/total surface area of the array) is relatively high (up to 90%), by leaving small spacing between individual elements. As the ultrasound power output of a transducer array is proportional to the surface area of PZT or PCC, the high packing density maximizes the ultrasound power output. However, PCC needs kerf gaps >0.5 mm between active elements for electric isolation to prevent arcing. This can account for a large fraction of the active area for arrays with small elements, thus the packing density for array with small elements (e.g., <5 mm) can be low (e.g., <60%).

Due to the delicacy of the traditional fabrication process, this method can only be performed in a labor-intensive manner. Device inconsistency due to variability of worker’s skill level has been a large barrier for mass production. The individual traditional elements are permanently embedded within the array and cannot be replaced. For a transducer array with multiple elements (e.g., hundreds or more), if certain elements are broken, either the array needs to be used at a reduced capacity or the entire array needs to be replaced. Given the high requirement of ultrasound power or pressure output histotripsy therapy, an array functioning at partial capability may result in incapability to treat certain patient populations or target tissues. Additionally, target tissues blocked by bones or gassy organs such as the lung can require an available acoustic window that may not be a typically geometrically symmetric shape suited for traditional fabrication.

This disclosure provides novel ultrasound transducer arrays including methods to design and fabricate an ultrasound array transducer with a high packing density and removable modular elements facilitated by rapid prototyping and arbitrarily shaped modular elements. The pre-designed arbitrary shaped elements can be made with rapid prototyping to maximally utilize the transducer surface area. Each of these individual elements can be built into a removable element by cutting a ceramic material to the pre-designed shapes, bonding the elements to 3D printed backing and matching layers, and then coating the elements in a thin high dielectric strength film. This coating is configured to insulate each element, while minimize the spacing between removable element modules. The element modules can then be assembled to a transducer scaffold.

The ultrasound transducer arrays of the present disclosure provide the following benefits and advantages over traditional transducer arrays: 1) High packing density - the techniques, systems, and methods described herein provide transducer arrays with a high packing density (e.g., area occupied by PZT or PCC/total array surface area > 90%). In comparison, traditional transducer arrays typically have a packing density below 70%, 2) Transducer element shape - the techniques, systems, and methods described herein provide the use of multiple pre-designed arbitrary shapes of elements to maximally utilize the transducer surface area. For example, the surface area of the transducer array can be divided into multiple concentric rings, and each ring can then split into trapezoidal shaped elements of equal area, while the trapezoidal elements at different rings may be of different sizes, 3) Array geometry - the techniques, systems, and methods described herein provide customized array geometries based on the available acoustic window for a given target tissue without blockage from bones or gassy organs. The electric steering range and the aberration of acoustic propagation through the acoustic path for a given target tissue can further be used to determine the individual transducer element sizes, 4) Fabrication - the techniques, systems, and methods described herein provide a novel way of enclosing and electrically insulating transducer elements of the transducer array. For example, the PZT or PCC individual transducer elements can be bonded to 3D printed backing-mounts and front matching layers of the same shape, such that the profiles of the stacking and bonding element-components are flush. Then the element stack can be coated by a thin layer of epoxy (e.g., 125-microns thick) with high dielectric strength (e.g., 1-2 kV per 25 microns at 75-microns thickness) to serve as a very thin electrical insulator. Without depending on a modular housing wall to isolate each element, the spacing between elements can then be significantly reduced, and the packing density of the array can be increased.

The present disclosure provides simulation algorithms to enable selection of the optimized frequency, array geometry, and element geometry to minimize aberration, maximize focal pressure, and achieving a sufficiently large electric steering range to maximize the treatment speed. In one implementation, the simulation tool is built on a large database of historical patient imaging, such as patient MRI/CT scans. The simulation tool can further utilize previous transducer data, material data, and tissue testing data.

### Introduction

Transcranial magnetic resonance guided focused ultrasound (tcMRgFUS) are provided herein for noninvasive ablation to treat neuro-disorders and brain tumors. Guided by MRI, ultrasound is applied from outside the skull and focused on the target brain tissue to produce thermal ablation, while the surrounding brain and the skull remain intact. Commercial tcMRgFUS systems have been approved by the U.S. Food and Drug Administration (FDA) to treat essential tremor by ablating a single focal volume within the central nervous system. Clinical trials on using tcMRgFUS to treat Parkinson’s diseases are also currently ongoing. However, due to overheating of the skull, which is highly absorptive and reflective of ultrasound, it is challenging for tcMRgFUS to treat locations within 2 cm from the skull surface, rendering inoperable up to ~90% of the cortex where brain tumors often reside. Moreover, the peri-target heating due to the ultrasound absorption in surrounding tissue limits the treatment rate of tcMRgFUS for a volume target, resulting in long treatment time that may be unbearable for patients with large tumors.

Unlike tcMRgFUS that relies on heating produced by continuous sonication, histotripsy uses short (several µsec), high pressure ultrasound pulses (>26 MPa) to generate focused cavitation bubbles, which mechanically fractionate and liquefy the target tissue into acellular homogenate. With long cooling times between pulses (ultrasound duty cycle <0.1%), transcranial histotripsy reduces heating of the skull and surrounding tissue while effectively ablating the target tissue. Gerhardson et al. have shown that applied through excised human skulls, histotripsy can liquefy up to 40 ml of clot within 20 minutes (corresponding to a treatment rate of 2 cm³/minute) in a wide range from the skull base to 5 mm from the inner skull surface, while keeping the temperature increase in the skull <4° C. As histotripsy mechanically disrupts the target tissue, there was a concern that histotripsy may cause excessive hemorrhage or edema in the brain. An initial in vivo study by Sukovich et al. showed that cerebral lesion can be generated in the normal pig brain without excessive bleeding in the acute and subacute phases after treatment. These preliminary results suggested the potential of using histotripsy for non-invasive transcranial treatment.

Since histotripsy-generated cavitation can be clearly visualized by ultrasound imaging, histotripsy treatment is typically guided by ultrasound imaging. However, transcranial ultrasound imaging remains as a challenge without contrast agents. To develop transcranial histotripsy techniques for non-invasive brain applications eventually, transcranial MR-guided histotripsy (tcMRgHt) is necessary to provide MR brain scans for targeting and monitoring to ensure treatment accuracy. Previous studies by Allen et al. have shown that histotripsy-induced cavitation and tissue fractionation can be visualized on MR with specialized MRI sequences.

Here, the first tcMRgHt system is designed and fabricated. Although the feasibility of transcranial histotripsy and MRI guidance has been shown separately, developing an integrated tcMRgHt system presents a substantial technical challenge. The design of tcMRgHt system is different than general histotripsy systems mainly in three aspects. 1) The system is required to be MR-safe with minimal mass of metal, and the cables should be long enough to ensure the driving electronics and power supplies are outside the 0.5 mT line, often conveniently in the MR control room separately from the scanner room. 2) Sufficient MR image quality is required with the tcMRgHt system in the MR scanner for treatment targeting and monitoring. Therefore, noises and artifacts introduced by the histotripsy system need be mitigated by appropriate separation and filtering of the electronics from MRI receive coils and possibly careful synchronization between the histotripsy pulses and MRI RF sequences. In addition, our design goal includes sufficient headroom of ultrasound pressure and a large electronic focal steering range to maximize the treatment efficiency for volume targets in the brain. These features all need to be taken into consideration and carefully addressed when developing this tcMRgHt system.

In this disclosure, a tcMRgHt system is provided which includes a 700-kHz, 128-element MR-compatible ultrasound phased array. The tcMRgHt system is acoustically characterized with focal pressure output, beam profiles, and electronic steering profiles. The MR-compatibility of the system can be quantitatively assessed by signal-to-noise ratio (SNR), B0 field map, and B1 field map acquired using gradient echo sequences with the tcMRgHt system in a clinical 3T MRI scanner. A workflow for using the tcMRgHt system to deliver a transcranial histotripsy treatment is provided.

### Histotripsy System Design and Fabrication

### Array Design

The structural design of the new MR-compatible transcranial transducer was based on previously developed hemispherical arrays with a focal distance of 150 mm. The center frequency of 700 kHz was chosen based on an optimization taking into account the skull transmission, aberration, focal gain, electronic steerability, cost, and electrical component limitations. The full hemispherical array includes 360 17-mm square modules, resulting in a surface area packing density of 74% compared to 57% for the previous 500 kHz 256-element design. The increased frequency and packing density of this new design enables a significantly higher power output and electronic steering range above the intrinsic cavitation threshold through a skull.

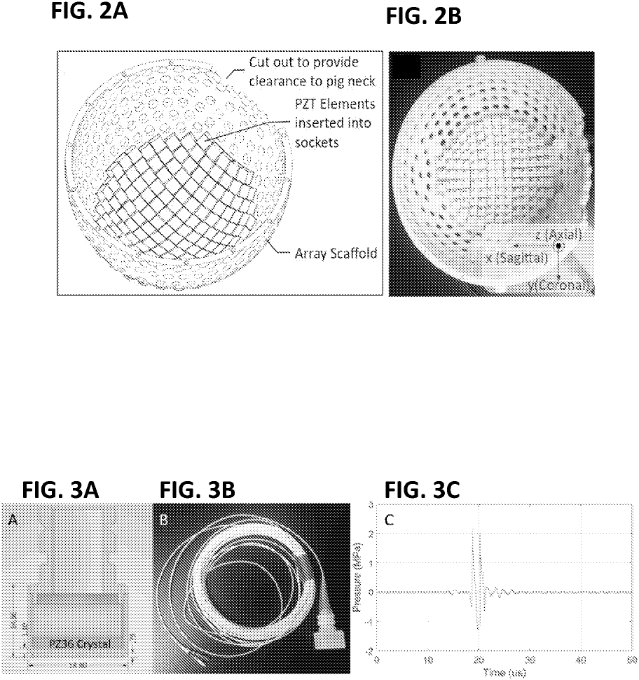

The initial motivation for this tcMRgHt system is to enable preclinical studies in the in vivo porcine brain trough an excised human skull, which is a previously used model for the tcMRgFUS studies. The pig skull is much thicker than a human leading to excessive attenuation, therefore, a craniotomy was first performed to create a 60 mm diameter opening through the pig skull to access the pig brain. An excised human skullcap was then placed over the porcine brain. With this experimental setup, approximately ⅔ outer portion of the 360-element array aperture would be blocked by the remaining pig skull. Thus, for the purpose of the porcine study, we used the 360-element array scaffold but only populated the inner ⅓ portion of the full array scaffold for this tcMRgHt system, resulting in an effective aperture of the array truncated to the inner 128-elements with an effective f-number (focal distance/aperture diameter) of 0.74 as demonstrated in FIGS. 2A-2B. FIG. 2A provides a schematic drawing of the array and FIG. 2B shows a photo of the 128-element MR-compatible histotripsy array.

For the design, a linear simulation program was used to simulate the pressure output and electronic focal steering range. The simulation was calibrated using the experimentally measured output of a single module driven at 3.5 kV in the free field and through the skull. With the 700 kHz 128-element array design, the maximum pressure achieved in free field, through the skull with aberration correction, and through the skull without aberration correction was estimated to be 116, 102, and 51 MPa, respectively. The simulated -6 dB steering range in free field was 24 mm in lateral direction and 42 mm in axial direction.

### Transducer Fabrication

The 128-element array can be fabricated by mounting 128 individual element modules to a transducer scaffold housing. Each module can be constructed from a porous PZT material (PZ36, Meggitt A/S, Denmark) in a 3D printed housing. FIGS. 3A-3C show a cross-section of transducer module design and photo of an element fabricated in house. Two matching layers with the thickness of a quarter-wavelength were used to provide a smoother gradient for acoustic impedance than a single matching layer, so that acoustic energy transmits more efficiently from the PZT to the medium. The backing end of the module is filled with marine epoxy, which ensured a water-tight seal around the PZ36 element and allowed the module to be fully submerged in the water-based propagating media. O-ring retaining grooves on the outside of the housing allowed the modules to be secured to the sockets on array scaffold via an easily removable O-ring and to be easily replaced individually if any module is damaged, thus keeping maintenance costs low. Electrical connections to the individual modules were made via micro-coaxial cable and high-density connectors (DL2-96, ITT Cannon LLC, Irvine, CA, USA). 3D-printed strain reliefs were attached around the connection to provide tension relief for the cables.

FIG. 3A illustrates one embodiment of a single transducer module, including a cross-section of the transducer module design. The PZ36 crystal is labeled in FIG. 3A. Two matching layers can be placed in front of the crystal to provide a smooth gradient for impedance matching. FIG. 3B is a photo of a single transducer module with 38-feet coaxial cable. FIG. 3C shows the pressure waveform produced from a single module at 150 mm in free field with a drive voltage of 3.5 kV. The peak negative pressure was 1.31 MPa.

### Driving Electronics

Driving electronics were built in-house to produce 3.5 kV peak amplitude and 20A to pulse each transducer element to enable the generation of microsecond length (1-cycle at 700 kHz) ultrasound pulses at a very focal high pressure for histotripsy. The transducer elements are connected to driving channels via high-density connectors. A FPGA (field-programmable gate array)-based microprocessor controlled via USB by MATLAB interface allows arbitrary pulsing of modules with 10 ns timing precision. Programmable trigger signals can be fed from MR scanner or sent to the scanner to synchronize the histotripsy pulse sequence with imaging sequence. 1-cycle pulse length will be used to generate cavitation, as this extremely short pulse length allows us to generate cavitation close to the inner skull surface without forming pre-focal cavitation at the skull surface or forming standing waves in the brain.

### Compatibility With MRI System

The new array adheres to standards for a MR conditional device to ensure patient safety and good imaging quality. All metallic components (fasteners, cables, etc.) consist of non-ferrous materials. With nylon scaffold, 3D printed housing and matching layers, and sintered silver electrode PZT crystals, this design minimizes the mass of metal to be imaged to maintain good MR image quality. The modular construction technique used for our system inherently results in a segmented ground plane, which has been shown to yield much better MR image quality compared to continuous plane design, as it allows RF magnetic field penetration through spaces between the ground plane segments. The driving electronics were placed in an adjacent control room outside of the MR scanner room, and the coaxial cables were fed through the waveguide penetrations of the panel between control room and scanner room for shielding. All experiments were done with a clinical 3T MRI scanner (Discovery MR750, GE Healthcare) in the Functional MRI Laboratory located adjacent to our histotripsy lab.

### Support Structures

To facilitate the in-vivo transcranial pig treatment, support structures are required to: 1) stably mount the transducer array onto the MR scanner bed; 2) firmly fix an excised human skull at a set location and orientation; and 3) position pig heads of a range of sizes at a desired location and orientation. The assembly of the transducer and all its support structures is illustrated in FIGS. 3A-3C. The bottom of the array frame can be contoured to mate with the scanner to make the array stay steady on the MR bed. The whole assembly was placed in a cylindrical waterproof canvas bag and immersed in the coupling media during the experiments.

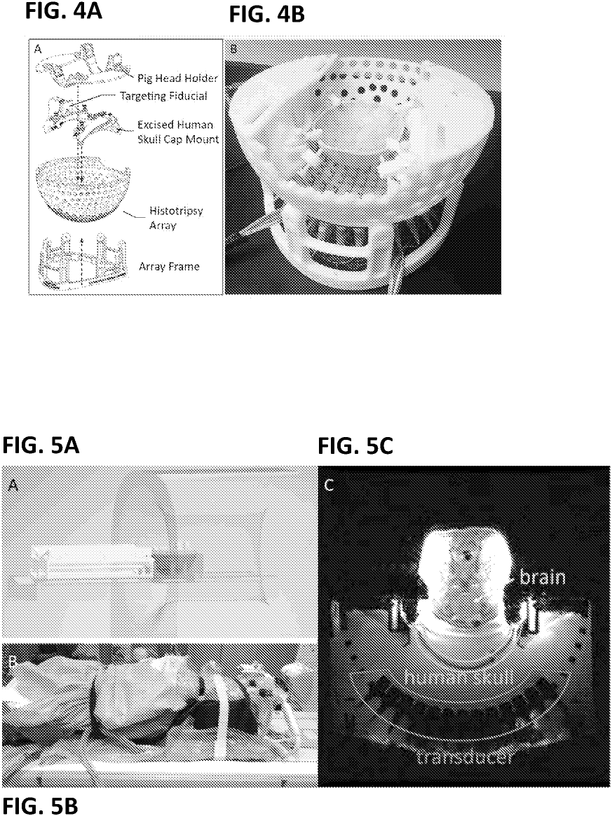

FIGS. 4A-4B show an exploded view and photo of a histotripsy array and accessories according to some embodiments of the disclosure. As shown in FIG. 4A, the array can include an array frame, a histotripsy array, and optionally, for animal studies or clinical trials, a skull cap mount and an animal holder (e.g., pig head holder).

The skull cap mount or skull holder is designed to be mounted to the unpopulated spaces on the scaffold and was shaped to avoid interfering with the ultrasound propagation path from any of the populated transducer elements at the bottom of the scaffold. To accommodate a variety of skull cap sizes and shapes, adjustable screws and clamps were used to fix the skull around its perimeter. Fiducial markers were also attached to the skull holder to quickly identify the geometric focus of the transducer array from an MR image and assist in targeting.

For the animal holder, neck and snout holders were designed using empirical measurements from a couple of pigs of representative size (60 lbs. and 70 lbs. juveniles) for our experiments, and the heights of these holders were arranged to hold the pig head level and at an optimal angle to provide the best acoustic window through the craniotomy opening. Adjustable Velcro straps fitted to the holders were used to secure the pig’s neck and snout during the experiments. These straps also provided some level of vertical adjustment for pig head positioning. Additionally, the neck and snout holders were fixed to a series of slots on a top plate that was fabricated using acetal plastic. These slots allowed adjustment to accommodate different pig head lengths as well as forward-to-rear and left-to-right positioning of the pig head. The plates also provided support for the MR surface coil pads, reducing any chances for them to shift or tilt during the image acquisition.

### Transcranial Acoustic Characterization

The fully assembled MR-compatible histotripsy array was acoustically characterized in free-field and through an excised human skullcap to determine the focal pressure, the size of focal zone, and the capability of electronic focal steering. The pressure field profiles were obtained in degassed water at room temperature and validate with the simulation results.

### Skull Preparation

For experimental purposes, an excised human skullcap can be obtained and used for the phantom and in vivo porcine experiments. The skullcap can be de-fleshed and cleaned after extraction and kept in either water or a 5% bleach-water solution thereafter. The bleach was added to prevent the growth of algae or bacteria on the surface of the skullcap and the walls of the storage container. Before experiments, the skullcap was degassed in water inside a vacuum chamber for a minimum of 6 hours. During the experiments, the skullcap was held by the skull holder and fixed at an identical location and orientation throughout all experiments including acoustic characterization, MRI testing and pig treatment. The major dimensions of the skullcap were 158 mm on the long axis (maximum front-to-back length on the exterior surface), 139 mm on the short axis (maximum end-to-end length on the exterior surface), and 56 mm in depth (maximum distance from the interior surface to the cut plane of the skull). The minimal and maximal thicknesses of the skullcap were 2.5 mm and 8.5 mm respectively. The transcranial attenuation was characterized to be 72% as the average reduction in the peak-negative pressure amplitude of the histotripsy array through the skullcap compared to the free field at the geometric focus of the transducer, when the free-field P- amplitude was measured to be 8~15 MPa.

### Focal Pressure

To characterize the focal pressure of the tcMRgHt transducer array, the peak-negative pressure (P-) at the geometric focus was measured in free field and through the skullcap using a fiber-optic hydrophone up to 15 MPa. At P- higher than 15 MPa, the pressure could not be measured directly due to instantaneous cavitation generation at the fiber tip. In this case, an extrapolated estimate of the focal pressure was obtained using a calibrated bullet hydrophone (HGL-0085, Onda, Sunnyvale, CA, USA) with each element fired individually. The P- was estimated as the linear summation of peak negative pressure from all elements up to the highest driving voltage of 3.5 kV. This operation effectively aligned the waveform from all elements and compensated for the phase aberration through the skull. The P- generated with 3.5 kV driving voltage in free field and through the skull without aberration correction were estimated from linear extrapolation of data measured by the fiber-optic hydrophone.

### Beam Profiles

To characterize the dimensions of the focal zone, 1-D beam profiles around the geometric focus were obtained in free-field and through the skullcap. Beam profiles were measured at low pressure (i.e., <2 MPa) using a needle hydrophone (HNR-0500, Onda, Sunnyvale, CA, USA) affixed to a motorized 3-D positioning system. The hydrophone was first positioned at the geometric focus and then scanned ± 20 mm from the focus in 0.25-mm steps along the sagittal, coronal and axial axes of the skullcap. As this tcMRgHt system delivers 1-cycle pulses for intrinsic threshold histotripsy, beam profiles were acquired in reference to P- at each hydrophone position. The focal size was determined as full width half maximum (FWHM) of the focal beam profile on three axes.

### Electronic Focal Steering Range

To characterize the electronic focal steering range, the focal pressure as a function of electronic focal steering location was measured in free-field and through the skullcap with a fixed driving voltage. The array was steered in 0.25-mm steps across a ± 25-mm range of locations centered at the geometric origin in the sagittal, coronal and axial dimension. For each electronic focal steering location, the 3-D positioner moved the needle hydrophone (HNR-0500, Onda, Sunnyvale, CA, USA) to the current electronic focal steering position to record the pressure. To ensure the hydrophone was not damaged, the array was run at a low-pressure amplitude for these measurements (i.e., <2 MPa). As this approach measures the pressure generated by all elements pulsing simultaneously, for the transcranial measurement it includes both the amplitude attenuation and phase aberration induced by the skullcap. To characterize the transcranial electronic steering range with phase aberration correction, a calibrated bullet hydrophone (HGL-0085, Onda, Sunnyvale, CA, USA) was used to measure the pressure generated by each element individually through the skullcap. The peak negative pressures from all elements were summed together as the pressure with aberration correction. The electronic steering range was characterized by the -6 dB range and the effective therapeutic range where >26 MPa can be generated, as the intrinsic cavitation threshold in brain tissue is 26 MPa.

### Lesion Generation With Electronic Focal Steering

To demonstrate transcranial histotripsy using electronic focal steering only, lesions were generated in a red blood cell (RBC) phantom through the human skullcap. The RBC phantom consisted of 2 layers of transparent agarose gel and a thin layer of gel with 5% bovine red blood cells in-between. As the RBC-gel layer changed from translucent and red to colorless after the destruction of the RBC due to cavitation, the phantom provided good contrast for visualizing cavitation damage. Lesions were generated in a sparse grid pattern to show the precision of histotripsy ablation and in a continuous grid to demonstrate volume treatment. The sparse grid treatment was applied through the skull without aberration correction, while the volume treatment was done with phase aberration correction using cross-correlation. Two hundred histotripsy pulses were delivered to each steering location through the skullcap at a pulse repetition frequency (PRF) of 20 to 50 Hz. The tcMRgHt system was fired at a fixed acoustic power which provides p- of 50 MPa at the geometric focus of the array with aberration correction.

### MRI With Histotripsy System

The MR-compatibility of the tcMRgHt system was evaluated for noise and artifacts induced by the experimental setup. The body coil of MRI scanner was used for both transmission and receiving in this section. Sound was coupled with degassed deionized water and degassed saline from the histotripsy transcranial array through the excised human skull, for SNR measurement and field maps respectively.

### Signal-to-Noise Ratio (SNR)

SNR has traditionally been presented as an important index of image quality in MR scans, which compares the level of a desired signal to the level of background noise. To understand the main source of noise in the tcMRgHt system, we measured the SNR in four cases: 1) cables are left inside the scanner room; 2) cables are placed in the control room through the wave guide penetrations, but not connected to the drive electronics; 3) cables are connected to the drive electronics via high-density connector in the control room and the electronics are powered up; 4) ultrasound array is pulsed at 10 MPa below cavitation threshold at a PRF of 10 Hz. A 2D spoiled gradient echo sequence was used for imaging with the following imaging parameters: TE= 10 ms, TR= 500 ms, flip angle = 30°, NEX = 2, grid size = 256 × 256 (re-sampled to 512×512), FOV=38 × 38 cm², slice thickness = 3 mm. The SNR for each case was calculated as µ/σ, where µ was the mean of 100×70 voxels in focal region of the transducer and σ was the standard deviation of the 400×80 background voxels in the deionized water.

### B0 Homogeneity

Field homogeneity refers to the uniformity of a magnetic field in the center of a scanner when no patient or phantom is present. The main magnetic field (B0) homogeneity is critical for MR image quality, as poor field homogeneity causes artifacts such as image distortions, blurring, and signal loss. Ideally, the B0 is expected to be homogeneous within a specified tolerance, and the off-resonance effects can be mitigated by shimming operation prior to the scan. However, the field can be further distorted by any wires, metal or fringe fields in the immediate environment of the scanner.

To investigate the B0 homogeneity with the presence of the tcMRgHt system in the MR scanner, we measured the off-resonance in Hz as a B0 map from two scans with different echo time using a standard method^{1,2}. The mean and standard deviation of 60×25 voxels in the focal region of histotripsy array were used as metrics for off-resonance induced by tcMRgHt system. A 2D spoiled gradient echo sequence was used for imaging with the following imaging parameters: TE₁= 10 ms, TE₂= 12 ms, TR= 500 ms, flip angle = 30°, NEX = 1, grid size = 192 × 192 (re-sampled to 512 ×512), FOV=38 × 38 cm², slice thickness = 3 mm.

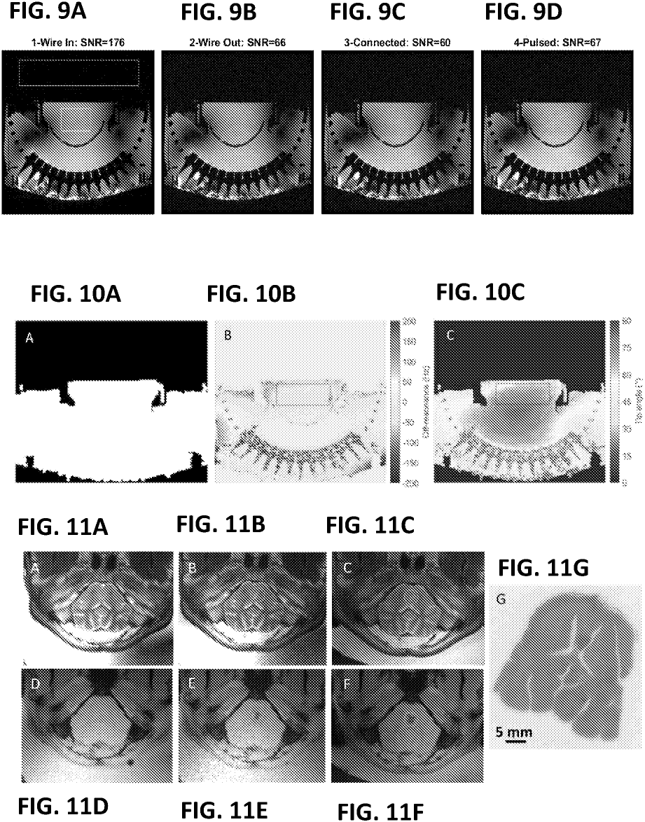

For field homogeneity measurements, the water bucket was filled up with 0.2% phosphate buffered saline solution instead of water, to mimic the in vivo pig treatment setup when saline is used as coupling media. An intensity-based binary mask shown as FIG. 9A was applied to the reconstructed B0 and B1 data to mask out the air-filled areas, as we are most interested in the field homogeneity within the histotripsy system.

### B1 Homogeneity

The radiofrequency (RF) field (B1) is applied perpendicular to the main magnetic field (B0). For in vivo MRI at high field (>=3 T), it is essential to consider the homogeneity of the B1 field. When exciting a large collection of spins, inhomogeneity in B1 results in nonuniform tipping of spins, leading to spatially varying image contrast and artifacts. The B1 field experienced by spins within the object is influenced by several factors including the distance from the RF transmit coil to the object, dielectric properties of the object, and factors related to the object size and RF wavelength.

To investigate the B1 homogeneity with the presence of tcMRgHt system in the MR scanner, we measured the actual tip angle using the double angle method. Since the actual tip angle is proportional to the B1 magnitude, the mean and standard deviation of 60×25 voxels in the focal region were used as metrics for B1 inhomogeneity induced by tcMRgHt system. A 2D spoiled gradient echo sequence was used for imaging with the following imaging parameters: TE= 10 ms, TR= 6 s, α₁= 60°, α₂= 120°, NEX = 1, grid size = 192 × 192 (re-sampled to 512 ×512), FOV=38 × 38 cm², slice thickness = 3 mm.

### In Vivo Treatment: Pilot Study on Transcranial Pig Brain Ablation

The feasibility of the tcMRgHt system was demonstrated in the in-vivo porcine brain through the excised human skull in two 60-80 lbs. adolescent pigs. For each pig, a 60 mm diameter circular region of the skullcap was surgically removed with the dura kept intact and then the skin sutured closed. This was done 2 days prior to histotripsy treatment to allow any air trapped in the incision to be absorbed.

On the treatment day, the pigs were anesthetized, and the vital signs were monitored through the treatment process. The experimental setup in the MR scanner is shown as FIG. 5. Sound was coupled with degassed saline from the histotripsy transcranial array through the excised human skull, sutured skin, and dura into the pig brain. The pig was placed supine on a v-tray with its head supported by snout and neck holders. The tcMRgHt system and the pig were placed on the MRI bed and imaged by MRI prior to the treatment. The histotripsy treatments consisted of 50 pulses per location in a targeted volume in a range of 27 to 108 mm³, at a pulse repetition frequency of 10 Hz at a p- of 51 MPa. For the first pig, a 3×3×3 mm lesion was generated applied with a spacing of 1 mm on 3 axes between adjacent focal positions. A 6×6×3 mm lesion was generated in the second porcine brain, with a spacing of 1 mm on x- and y-axis and a spacing of 1.5 mm on z-axis.

FIGS. 5A-5B illustrate a diagram and photo, respectively, of the experimental setup for in vivo pig treatment. The tcMRgHt system was placed on the MR bed. The pig lied on its back and was stabilized on top of the v-tray, with the pig head supported by the snout and neck holder on the tcMRgHt system. MR images on transverse plane (C) demonstrate the positions of transducer elements, human skull and pig brain.

The MRI treatment set-up used 10 cm DuoFLEX receive array coils. During the treatment, a diffusion-weighted RF pulse sequence was used to detect histotripsy-induced cavitation on MRI. In order to localize cavitation during treatment, the RF pulse sequence was synchronized with histotripsy pulses such that the acquired image becomes sensitive to water displacement caused by cavitation. T2 and T2* images were acquired pre-treatment, immediately after the treatment, and 2~4 hours after the treatment. For the pilot study, the pigs were euthanized after post-treatment imaging was done.

### Results

### Focal Pressure

The maximal focal pressure achieved was measured to be 120 MPa in the free field, 72 MPa through the excised human skull without aberration correction, and 51 MPa through the skull with aberration correction, summarized in Table 1. Since the intrinsic cavitation threshold in the brain tissue is 26 MPa, the achieved focal pressure provided sufficient acoustic power for transcranial treatment.

TABLE 1

Measured pressure, focal size and steering range in three different cases

| | Maximal focal pressure (MPa) | Focal size (mm³) | Steering range metric | Electronic steering range | | |
|---|---|---|---|---|---|---|
| | | | | X (mm) | Y (mm) | Z (mm) |
| FF | 120 | 1.9×2×7.6 | -6 dB | 26.5 | 26.5 | 50 |
| | | | >26 MPa | 50 | 50 | 50 |
| TC with AC | 72 | 2×2.2×7.5 | -6 dB | 28 | 27 | 50 |
| | | | >26 MPa | 37 | 35 | 50 |
| TC without AC | 51 | 2.3×2.2×7.9 | -6 dB | 27 | 26 | 50 |
| | | | >26 MPa | 26 | 25.5 | 50 |

### Beam Profiles

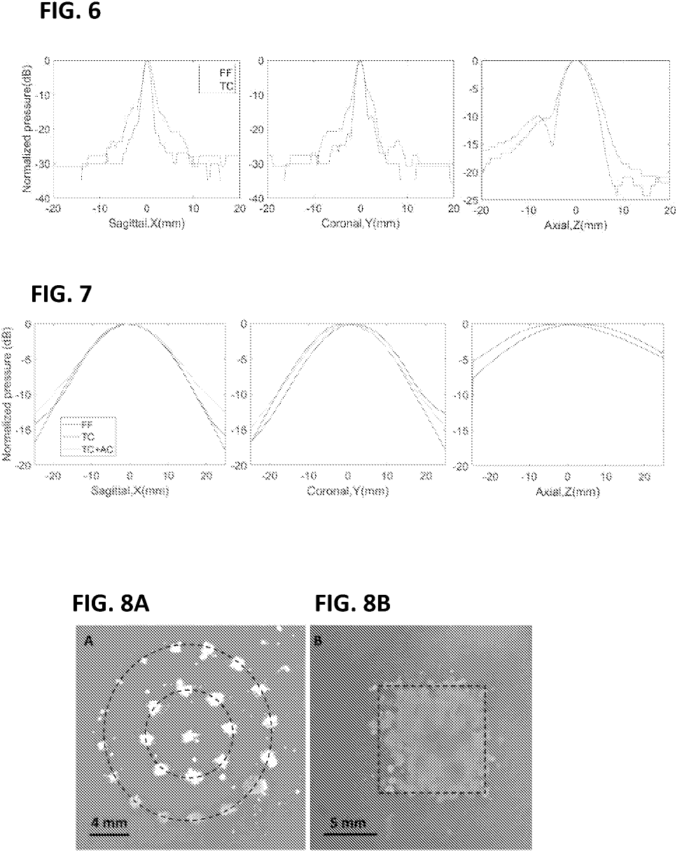

1-D beam profiles around the geometric focus are shown in FIG. 6. The focal size was determined to be 1.9×2×7.6 mm³ in free field, which is smaller than the focal zone through the skull on the transverse plane. In all cases, the axial length of the focal zone is about 3 times larger than the lateral size. The beam profiles of the tcMRgHt array include FF: free field; TC: through the human skullcap.

### Electronic Focal Steering Range

The normalized peak-negative pressure as a function of electronic steering location along each axis was summarized in Table 1. As shown in FIG. 7, the -6 dB range in three cases remained almost identical, but the effective therapeutic range (where p->26 MPa can be achieved) through the skull was significantly smaller than that in free field, because the attenuation and aberration induced by the skullcap became prominent as the steering locations move further from the geometric focus. The effective therapeutic range was determined to be 25.5 mm laterally and 50 mm axially through the skull without aberration correction, and 37 mm laterally and 35 mm axially through the skull with aberration correction.

FIG. 7 illustrates steering profiles of the tcMRgHt array. FF: free field (blue); TC: through the human skull (red); TC+AC: through the human skull with phase aberration correction (green).

### Lesion Generation With Electronic Focal Steering

The transcranial treatment using electronic focal steering only was visualized using the RBC phantoms in FIG. 9. The size of lesions on the sparse circular pattern (FIG. 8A) ranged from 0.6 mm to 2.3 mm due to the aberration and attenuation through the excised human skull, suggesting the precision of histotripsy treatment. The 10-mm continuous square lesion in FIG. 8B demonstrated that tcMRgHt system can treat volume target using electronic focal steering only through the excised human skull.

FIGS. 8A-8B shows histotripsy ablation generated by electronic focal steering in the RBC phantom. FIG. 8A shows a sparse circular pattern centered at geometric focus. FIG. 8B shows a 10-mm continuous square lesion representing a volume ablation zone in transverse plane.

### MRI With Histotripsy System

### SNR

FIGS. 9A-9D shows the images acquired from four cases described above and their corresponding SNR. The SNR decreased remarkably when cables were moved from the scanner room to the control room. For the treatment setup with cables outside the scanner room, the SNR when tcMRgHt system is idle or running are almost identical, indicating that the electrical excitation of histotripsy array negligibly interfered with the MR scanner, thus enabling sufficient SNR for targeting, treatment monitoring and post-treatment imaging.

FIGS. 9A-9D also show magnitude images from four experimental setup cases. The signal region and background region used for SNR measurement are shown in FIG. 9A. The measured SNR results are labeled above images correspondingly.

### B0 Homogeneity

FIG. 10B shows the measured B0 map. Artifacts occurred locally at the ultrasound transducer elements and did not extend into surrounding areas. In the focal region of histotripsy array, the mean and standard deviation of off-resonance was -16.3 Hz and 23.4 Hz, separately. Overall, the mean and standard deviation of off-resonance on the B0 map was -7.0 Hz and 44.8 Hz separately, suggesting a sufficient B0 homogeneity with the tcMRgHt system in the scanner.

FIGS. 10A-10C show. B0 and B1 field maps for tcMRgHt experiments. FIG. 10A shows binary mask applied to reconstructed field maps. FIG. 10B shows B0 field map for off-resonance in Hz. FIG. 10C shows a B1 field map for actual tip angle.

### B1 Homogeneity

FIG. 10C shows the measured tip angle map. Like the B0 map, artifacts occurred around the transducer elements but well-confined within the elements and array scaffold. Compared to the prescribed tip angle of 60°, over-tipping was shown in the central region of the image, coinciding to the effects previously seen in clinical human scans with the center being brighter than the edges. In the focal region of histotripsy array, the mean and standard deviation of flip angle was 69.1° and 16.0°, respectively. Overall, the mean and standard deviation of off-resonance on the B1 map with mask was 58.9° and 12.9° separately, suggesting a sufficient B1 homogeneity with the tcMRgHt system in the scanner.

### In Vivo Treatment

The tcMRgHt system was used to successfully create lesions in the brain of two pigs through the excised human skullcap. T2-weighted MR images showed hyper-intense histotripsy ablation zones compared to the surrounding untreated tissue, as the cavitation generated by histotripsy liquefies the tissue (FIGS. 11A-11G). These hyper-intense regions were well confined within the targeted volume and did not demonstrate significant brain edema. T2* images, as a measure of iron and hemosiderin in the brain, demonstrated no excessive bleeding in peri-target zones post-treatment. Ablation zones in both pigs were identified in the thalamus region adjacent to the third ventricle. The effective ablation zones were also confirmed by histology, which revealed complete cellular disruption within the ablation zones and great correlation to the identified treatment zones on MRI.

Referring to FIGS. 11A-11G, MR images of the pig brain pre- and post- histotripsy treatment are shown. FIG. 11A shows T2, pre-treatment; FIG. 11B shows T2*, pre-treatment; FIG. 11C shows T2, immediately post-treatment; FIG. 11D shows T2*, immediately post-treatment; FIG. 11E shows T2, 2-hour post-treatment; FIG. 11F shows T2*, 2-hour post-treatment; FIG. 11G shows a histology slice corresponding to the MR images showing an ablation zone adjacent to the third ventricle. (The hemorrhage on the surface of the brain was remaining clot from the craniotomy and was not associated with the histotripsy treatment.)

### Workflow for Transcranial Treatment

The workflow of using the tcMRgHt system to deliver a transcranial treatment was summarized as below:
Set up - The histotripsy array was placed in the empty water bag on the MRI scanner bed. A spirit level was mounted on top of the histotripsy array to check the horizontal level. The cable bundle was fed through the waveguide on the penetration penal and connected to the drivers in the control room via the high-density connectors. Degassed water or saline was added in the water bag. The degassed skullcap was mounted to the skull holder with the screws tightened to fix the position and orientation of the skull.
Aberration correction - For the in-vivo porcine treatment, hydrophone-based aberration correction was implemented to compensate for the phase variance introduced by the skullcap before the treatment to improve the efficiency of histotripsy treatment. The phase correction terms can also be estimated prior experiments via simulation-based correction or analytical CT-based correction.
Pre-treatment scan - Acquired pre-treatment MRI scans for the experimental phantom or animal with histotripsy system power off.
Targeting - Pretreatment MRI scans were used to locate the target with regard to the geometric focus of the histotripsy array via fiducial markers. Then, electronic focal steering pattern was created to treat a volume of target locations.
Treatment delivery - Delivered transcranial histotripsy treatment using the pre-set parameters. Real-time MRI monitoring was applied using the cavitation-sensitive MR pulse sequences previously developed by Allen et al.
Post treatment scan - Acquire post-treatment MRI scans with histotripsy system power off to analyze the treatment outcomes. Histotripsy ablation zones typically appear as hyper-intense regions on T2-weighted MRI and hypo-intense regions on T2* MRI.

Although this workflow was originally developed for in vivo porcine treatment, it can be easily adapted to facilitate other ex vivo phantom treatment or in vivo preclinical treatment with minor modifications.

### MR-Compatibility Analysis

As mentioned above, the difference between SNR in various cases indicated that the cable location had the most significant impact on SNR. The SNR decreased remarkably when cables were fed through the penetration panel waveguide, which attenuates the frequency components below its cut-off frequency to prevent the external RF interference. When conductors like cables were introduced in the waveguide, the waveguide became a coaxial cable with no cut-off frequency and allowed noise entering the scanner room. Conceptually the waveguide is only for use with optical fibers, gas hoses, and fluid pipes, and any electrical cables should pass the penetration panel via filtered BNC, DB-25 and other connectors on the penetration panel. However, the common commercially available filters only have at most 50 pins, and even if hundreds of pins are available, connecting all channels of histotripsy system via the filters would be time-consuming. We further decomposed the source of noise by calculating the ratio of external interference from the cables through the wall to object noise. When cables are in the scanner room, SNR₁ = µ/σ₁, where σ₁ denotes the standard deviation of the inherent thermal noise. When cables are fed through the wall, SNR2 = µ/σ2, where σ₂ is the standard deviation of the inherent thermal noise pulse the shielding noise through the wall, i.e., σ₂ = σ₁ + σ_{wall}. Therefore,

$$\frac{{σ}_{w a l l}}{σ 1} = \frac{S N R 1}{S N R 2} - 1 \approx 1.5 ,$$

which implies that the noise due to RF shielding through the wall is about one and one-half times the inherent thermal noise, indicating that improved filtering of the conductors can still lead to improves image SNR.

When the cables were placed in the control room, the SNR was barely influenced by the power status and electrical excitation of the tcMRgHt system. We also note that the operation of the histotripsy array did produce obvious streaking of other image artifacts. This matched well with our intuition, as the resonance frequency of the transducers on histotripsy array is much lower than the resonance frequency of 3T scanner (700 kHz compared to 128 MHz), and histotripsy treatment is performed at even lower PRF in a range of 1-200 Hz.

The off-resonance in the B0 map can be further explained based on the susceptibility of the materials involved in tcMRgHt system. The water is diamagnetic (susceptibility χ < 0) while piezoceramic material in the transducer elements is paramagnetic (χ > 0), therefore, the magnetic flux lines shift from water into the piezoceramic material, creating a lower field at the thin layer of water near the surface of transducer elements and leading to negative off-resonance. Similarly, water is also diamagnetic relative to air, so the magnetic flux lines shift from water into air, creating a lower field in water at the water-air interface. This explains the negative off-resonance on the water-air interface at the top and the bottom of the water bucket. The off-resonance at water-air interface was measured to be smaller than that on the transducer surface, which coincides with the fact that air is less paramagnetic compared to piezoceramic material. Conversely, the positive off-resonance on the left side of the image was identified to be a cable bundle, because the cables (copper) are moderately diamagnetic, and this leads to distortions in the nearby magnetic fields.

In general, the image quality achieved in this study was sufficient to demonstrate that ablation was successfully generated during histotripsy treatment. To further demonstrate the treatment outcome and evaluate the treatment safety, the imaging protocols can be optimized to achieve better resolution and SNR by using a larger number of averages, thinner slices, and a smaller FOV, etc. However, changing these parameters will also lead to longer acquisition time and risks of aliasing. More investigation will be done to realize a better trade-off between the image quality and other concerns.

### Next Generation of tcMRgHt System

The 128-element tcMRgHt system built in this study was specifically optimized for in vivo pig treatment. For human cadaver treatment or in real clinical cases, no craniotomy is needed as the ultrasound can propagate through the human skull. Therefore, to improve the treatment efficiency, a fully populated hemispherical histotripsy array can be used for cadaver and human studies. The steering profiles suggested that the acoustic power decreased with distance between the steering locations and the geometric focus. Therefore, for maximal acoustic efficiency, it is important to set the target tissue as close to the geometric focus of the histotripsy array as possible. However, placing the head at an appropriate location and orientation remained a major challenge, as observed during the in vivo pig treatment. A potential method to solve this issue would be to move the histotripsy array using a MR-conditional motorized positioner. A stereotactic frame to rigidly fix the patient head can also assist in co-registering the target to the histotripsy array and facilitate aberration correction. An acoustic coupler attached to the patient’s head can replace the water bag used in this study to ensure the ultrasound transmission from the array to the head. Modifications on support structures are also required to match the geometry of human head. These components will be incorporated into design for the next generation of the tcMRgHt system to promote the performance of transcranial treatment using MR-guided histotripsy.

Improvements regarding the drive electronics are also in the scope of our future work. Arcing on high density connector pins has been noticed at high driving voltage, which may cause crosstalk between channels and damage the transducer modules. Further investigation will be done on assessing the source of the arcing and mitigating such effect. Besides, the drive electronics for this system only included the transmission circuits, as our goal for this study was to demonstrate the feasibility of transcranial MR-guided histotripsy treatment. Involving receive circuits into the drive electronics will enable us to monitor treatment progress based on shockwave signal from acoustic cavitation.

### Real-Time Treatment Monitoring

For in vivo pig treatment in this paper, we achieved real-time monitoring using a specialized cavitation-sensitive MRI sequence. When synchronized with the histotripsy pulse sequence, the intra-voxel incoherent motion (IVIM) pulse sequence generated images showing the histotripsy ablation effect at the intended treatment location with temporal resolution of 2 second. This is the first study to show successful in vivo transcranial histotripsy guided by MRI. The real-time images were consistent with post-treatment images, but since this IVIM sequence was also T2*-weighted, it was difficult to distinguish between blood pooling and IVIM sources of signal loss. Besides, due to the large size of the water bucket, our FOV was set to 40 cm to prevent aliasing and our spatial resolution of 3.125 mm in plane for 5 slices. We plan to develop approaches to address both issues in the future.

### Conclusion

This study developed the first tcMRgHt system for in vivo treatment. A 700-kHz, 128-element MR-compatible phased array was designed and fabricated. Support structures were designed and constructed to facilitate the transcranial treatment. The MR-compatibility of the tcMRgHt system was assessed quantitatively using SNR, B0 field map, and B1 field map in a clinical 3T MRI scanner. Sufficient SNR and field homogeneity were achieved to provide good image quality for treatment evaluation and real-time monitoring. The tcMRgHt array was acoustically characterized with an estimated peak negative pressure up to 190 MPa in the free field, and 51 MPa through an excised human skullcap, and 72 MPa through the skullcap with phase aberration correction. The focal size of tcMRgHt array was measured to be 1.9×2×7.6 mm³ in the free field and slightly different through the human skull. The capability of electronic focal steering and precision of histotripsy treatment were visualized in a red blood cell agarose phantom. Using electronic steering only, the tcMRgHt system was able to create lesions in a range of 25.5 mm in the transverse plane and 50 mm in the axial plane through the human skull. The feasibility of transcranial tcMRgHt treatment was demonstrated by successfully generating ablation in the brain of two pigs through the excised human skull with no excessive hemorrhage or edema observed. These results demonstrated the feasibility of using this tcMRgHt system for in vivo transcranial treatment and enabled further investigation on MR-guided histotripsy.

What is claimed is:

1. A method of treating a patient with MR-guided histotripsy therapy, comprising the steps of:
identifying an ultrasound focal location of a histotripsy therapy transducer on a MR image;
positioning the ultrasound focal location on a target tissue;
transmitting histotripsy pulses from the histotripsy therapy transducer into the target tissue to generate cavitation in the target tissue;
acquiring MR images of the target tissue to monitor cavitation in the target tissue.

2. The method of claim 1, wherein identifying the ultrasound focal location comprises emitting ultrasound energy below a cavitation threshold from the histotripsy therapy transducer; and
detecting the ultrasound energy with a MR-ARFI system.

3. The method of claim 1, wherein detecting the ultrasound energy with the MR-ARFI system comprises detecting displacement at the ultrasound focal location.

4. The method of claim 1, wherein identifying the ultrasound focal location comprises emitting ultrasound energy to create a 1-4 deg C temperature increase at the ultrasound focal location; and
detecting the temperature increase with a MR thermometry system.

5. The method of claim 1, wherein the histotripsy pulses are transmitted through a skull of a patient.

6. The method of claim 1, wherein the target tissue is in a brain of a patient.

7. The method of claim 1, wherein acquiring images further comprises acquiring images of bubble expansion and collapse events and not the cavitation itself.

8. The method of claim 1, wherein acquiring MR images of the target tissue further comprises acquiring MR images with an intravoxel incoherent motion (IVIM) imaging pulse sequence.

9. The method of claim 8, wherein the IVIM sequence comprises a spin-echo (SE) sequence.

10. The method of claim 1, further comprising acquiring MR thermometry images of the target tissue to monitor heating of the target tissue.

11. The method of claim 10, wherein acquiring MR thermometry images is interleaved with acquiring MR images.

12. The method of claim 1, further comprising acquiring post-treatment MR images to evaluate histotripsy ablation.

13. The method of claim 12, further comprising quantitatively assessing a level of tissue disruption generated by histotripsy with the post-treatment MR images.

14. The method of claim 13, further comprising applying diffusion weighted MRI.

15. The method of claim 13, further comprising applying MR elastography.

16. The method of claim 13, wherein the acquiring MR images step is synchronized with the transmitting histotripsy pulses step.

17. An ultrasound system, comprising:
a histotripsy therapy transducer configured to transmit histotripsy pulses to an ultrasound focal location in a target tissue volume;
a MRI system configured to generate MR images of the target tissue volume, the MRI system being further configured to identify the ultrasound focal location of on a MR image of the target tissue volume, the MRI system being further configured to acquire MR images of the target tissue to monitor cavitation resulting from the histotripsy pulses in the target tissue.
